(19) United States
(12) Patent Application Publication
(10) Pub. No.: US 2023/0159447 A1
(43) Pub. Date: May 25, 2023

(54) CHEMOENZYMATIC PROCESS FOR COPRODUCTION OF A DISULFIDE AND A SULFOXIDE OR A SULFONE

(71) Applicants: Arkema France, Colombes (FR); Centre National De La Recherche Scientifique - CNRS, Paris (FR); Universite d'AIX-Marseille, Marseille Cedex 7 (FR)

(72) Inventors: Georges Fremy, Lacq (FR); Hugo Brasselet, Lacq (FR); Jean-Christophe Lec, Lacq (FR); Véronique Alphand, Marseille (FR); Katia Duquesne, Vitrolles (FR)

(73) Assignees: Arkema France, Colombes (FR); Centre National De La Recherche Scientifique - CNRS, Paris (FR); Universite d'AIX-Marseille, Marseille Cedex 7 (FR)

(21) Appl. No.: 17/801,109

(22) PCT Filed: Mar. 3, 2021
(86) PCT No.: PCT/FR2021/050362

§ 371 Date: Aug. 19, 2022

(30) Foreign Application Priority Data
Mar. 9, 2020 (FR) FR2002315

(51) Int. Cl. C07C 315/02; C12P 11/00; C07C 315/06

(52) U.S. Cl. CPC C07C 315/02; C07C 315/06; C12P 11/00

(57) ABSTRACT

Chemoenzymatic process for coproducing a disulfide and a sulfoxide or a sulfone The present invention relates to a chemoenzymatic process for coproducing disulfide and sulfoxide or sulfone from a composition M comprising: 1) a sulfide, 2) optionally an oxidizing agent, 3) an organic compound bearing at least one thiol group, 4) an enzyme E catalyzing the oxidation of said sulfide to sulfoxide or to sulfone, 5) an enzyme D catalyzing the formation of a disulfide bridge between two equivalents of said organic compound bearing at least one thiol group to form a dimer, and 6) a cofactor common to the two enzymes E and D; and also to a composition enabling especially the implementation of this process. The present invention also relates to the use of a mercaptan for reducing a disulfide bridge formed between two equivalents of an organic compound bearing at least one thiol group, and more particularly to the use thereof as regeneration substrate of the process described above.

### CROSS-REFERENCE TO RELATED APPLICATIONS

This application is a 371 filing of International Application No. PCT/FR2021/050362, filed Mar. 3, 2021, which claims priority to French Application No. 2002315, filed Mar. 9, 2020, the disclosures of these applications being incorporated herein by reference in their entireties for all purposes.

### FIELD OF THE INVENTION

The present invention relates to a chemoenzymatic process for coproducing disulfide and sulfoxide or sulfone from mercaptan and sulfide respectively, and to a composition enabling especially the implementation of this process. The present invention also relates to the use of a mercaptan for reducing a disulfide bridge formed between two equivalents of an organic compound bearing at least one thiol group, and more particularly to the use of a mercaptan as a regeneration substrate of an enzymatic cascade enabling the oxidation of sulfides.

### BACKGROUND OF THE INVENTION

Mercaptans are of great interest industrially and are currently in widespread use in the chemical industries, notably as starting materials in the synthesis of more complex organic molecules. For example, methyl mercaptan (CH₃SH) is used as a starting material in the synthesis of methionine, an essential amino acid used in animal nutrition. Methyl mercaptan is also used in the synthesis of dialkyl disulfides, in particular in the synthesis of dimethyl disulfide (DMDS), a sulfiding additive for hydrotreating catalysts for petroleum fractions, among other applications.

Mercaptans, and more particularly methyl mercaptan, are generally synthesized industrially by a known process starting from alcohols and hydrogen sulfide at elevated temperature in the presence of a catalyst according to equation (1) below:

$$\text{R-OH} \text{+} {\text{H}}_{2} \text{S} \to {\text{R-SH + H}}_{2} \text{O}$$

However, this reaction gives rise to the formation of by-products, such as the sulfides according to equation (2) below:

$$\text{R-OH} \text{+} \text{R-SH} \to {\text{R-S-R + H}}_{2} \text{O}$$

Mercaptans may also be synthesized from halogenated derivatives and alkali metal, alkaline earth metal or ammonium hydrosulfides according to equation (3) below (example given using a chlorinated derivative and a sodium hydrosulfide):

$$\text{R-Cl} \text{+NaSH} \to \text{R-SH} \text{+} \text{NaCl}$$

This second synthesis pathway also results in the presence of unwanted sulfides. Mercaptans, lastly, may be synthesized from olefins and hydrogen sulfide by acid catalysis or photochemically according to whether the target is a branched or an unbranched mercaptan, according to equation (4) below:

$${\text{R}}_{\text{A}} {\text{R}}_{\text{B}} {\text{C=CR}}_{\text{C}} {\text{R}}_{\text{D}} + {\text{H}}_{2} \text{S} \to {\text{R}}_{\text{A}} {\text{R}}_{\text{B}} \text{-CH-C} \left(\text{SH}\right) {\text{R}}_{\text{C}} {\text{R}}_{\text{D}}$$

Once again, this synthesis gives rise to sulfides as by-products.

These sulfides are obtained in high quantity industrially and are primarily sent for destruction. This represents an efficiency loss in the process for producing the intended mercaptan and an added cost associated with destroying them. Generation of wastes in this way is a real industrial problem for producers of mercaptans, who therefore look to derive value from these by-products. There are various ways of doing this.

First of all, there is a market for the sulfides themselves: dimethyl sulfide can be used as a food flavor or as an anticoking agent in the steam cracking of petroleum feedstocks. The demand in these markets, though, is very much lower than the amounts of sulfides produced.

The sulfides can also be converted to corresponding mercaptans by the sulfhydrolysis reaction. Nevertheless, the conditions required for carrying out this reaction are relatively harsh and give rise to new, parasitic reactions. This industrial application is therefore limited.

Finally, another means of deriving value from the sulfides produced involves the oxidation reactions of sulfides to convert them into sulfoxides and/or into sulfones. Chemical oxidation reactions of these kinds are well known. They entail different types of oxidizing agents such as sodium hypochlorite, hydrogen peroxide, oxygen, ozone or nitrogen oxides such as N₂O₄ in the presence or absence of catalysts.

As well as the chemical oxidations, sulfide oxidations may be catalyzed, in biological processes, by enzymatic catalysis in solution or in organisms, generally microorganisms.

Patent applications FR1906488 and FR1906489 respectively describe a selective process for preparing sulfoxides or sulfones from organic sulfides by enzymatic catalysis. The content of these two patent applications is incorporated here by reference in their entirety.

The processes described in these two patent applications thus enable sulfoxide or sulfone to be obtained selectively by using an enzyme catalyzing the oxidation of said sulfide to sulfoxide or to sulfone, according to the amount of sulfide present during the enzymatic reaction. When the sulfide is not wholly consumed during the step of conducting the enzymatic sulfide oxidation reaction, then the sulfoxide is selectively obtained. When the sulfide is wholly consumed during the step of conducting the enzymatic sulfide oxidation reaction, then the sulfone is selectively obtained.

The enzyme used may, however, require the use of a cofactor which must be able to be recycled (or regenerated) so that the enzymatic oxidation reaction can take place again.

This is because the cofactors, owing to their complexity and for economic reasons, are not generally added in stoichiometric quantities in industrial enzymatic processes. A variety of approaches have therefore been developed for recycling them and for regenerating the enzymatic cascades resulting therefrom:
the use of a molecule analogous to the cofactor. This approach involves using molecules that are simpler and hence less costly. However, the affinity between enzymes and cofactor that this produces is not high enough to consider industrial application and this approach represents an unacceptable cost for industrial processes for producing low added value products;
the use of enzymatic redox systems with use of a sacrificial substrate. These techniques generally necessitate the use of a second enzyme allowing the cofactor used to be recycled. The cost of the sacrificial substrate, though, greatly impacts the economic viability of such a process. This is especially true when sulfide is being oxidized to sulfoxide or to sulfone of low added value;
the use of whole cells. In this case it is the cellular machinery that regenerates the cofactor or cofactors used. The process is thus closer to fermentative processes: carbon-/hydrogen-containing molecules (such as glucose or glycerol) are generally added to enhance the performance characteristics of this system, and this represents an additional cost.

Consequently, a need exists for an industrially viable pathway for regeneration of the cofactors used in the enzymatic processes for the oxidation of the sulfides. There also exists a need for a process for deriving value from sulfides, especially those resulting from mercaptan synthesis, that is viable industrially and economically.

### SUMMARY OF THE INVENTION

An objective of the present invention is to meet all or part of the needs above. An objective of the present invention more particularly is to provide a pathway for regenerating (or recycling) the cofactor used for the enzymatic oxidation of sulfides to sulfoxides or to sulfones.

Another objective of the present invention is to provide a process for chemoenzymatic coproduction of sulfoxide or sulfone and disulfide.

A further objective of the present invention is to provide a process for chemoenzymatic coproduction of sulfoxide or sulfone and disulfide that is industrially viable, incorporating in particular a pathway for recycling cofactors that is simple, effective and economical.

Another objective of the invention is to propose a process allowing value to be derived from the sulfides produced during production of mercaptans, more specifically of methyl mercaptan.

The present inventors have found a, preferably selective, chemoenzymatic process for producing sulfoxide or sulfone that incorporates a system for recycling the cofactor of the enzyme catalyzing the oxidation of the sulfides, with added value.

The inventors, indeed, have surprisingly found a chemoenzymatic cascade which not only is compatible with the sulfoxide or sulfone production process but also allows the coproduction of a second product of interest, the disulfide, instead of using a sacrificial substrate. Hence it is the oxidation of a mercaptan to disulfide at the end of the cascade that will enable recycling of the cofactor used in the sulfoxidation.

For example, when the oxidizing agent is oxygen, the overall equation for the cascade may be written as follows:

$$\begin{matrix}\text{RSR} ′ + {\text{O}}_{2} + 2 \text{R} ″ \text{SH -> RS} \left(\text{O}\right) \text{R} ′ \text{+} {\text{H}}_{2} \text{O} \text{+} \text{R} ″ \text{SSR} ″ \text{, for sulfoxides} \\ \text{RSR} ′ \text{+} {\text{2 O}}_{2} + 4 \text{R} ″ \text{SH ->} \text{RS} {\left(\text{O}\right)}_{2} \text{R} ′ \text{+} {\text{2 H}}_{2} \text{O + 2 R} ″ \text{SSR} ″ \text{, for sulfones} \text{.}\end{matrix}$$

Hence the mercaptan may be considered a hydrogen donor.

The enzymatic cascade according to the invention proceeds more particularly as set out below.

In the presence of an enzyme catalyzing the oxidation of the sulfide to sulfoxide or to sulfone (hereinafter enzyme E), of its cofactor and of the oxidizing agent where appropriate, the sulfide is converted to sulfoxide or to sulfone and the reduced cofactor is regained in its oxidized form.

Then, in the presence of an enzyme catalyzing the formation of a disulfide bridge between two equivalents of an organic compound bearing at least one thiol group (hereinafter enzyme D) and of this organic compound:
said oxidized cofactor is reduced (and so recycled by virtue of the hydrogen atoms donated by the organic compound), and
a dimer is formed by a disulfide bridge between two equivalents of said organic compound (the organic compound passes from its reduced “monomer” form into its oxidized “dimer” form).

According to the invention, then, “organic compound” is understood to be a compound of formula R-SH and “dimer” of this compound is understood to be a compound of formula R-S-S-R (one well-known example is glutathione G-SH in dimeric form G-S-S-G, called glutathione disulfide).

The cascade then finishes by virtue of a chemical equilibrium: the oxidized dimer is reduced by reaction with two equivalents of mercaptan, which are converted to the corresponding disulfide.

The presence of mercaptan makes it possible, by virtue of a chemical equilibrium governed by Le Chatelier’s principle, to:
reduce the dimer to two equivalents of organic compound (hence the latter is also recycled due to the hydrogen atoms donated by the mercaptan); and
form a disulfide.

It is the enzymatic reaction that displaces the chemical equilibrium toward the formation of the disulfide.

The mercaptans may be used in stoichiometric quantities relative to the sulfides.

More particularly, the formation of one sulfoxide equivalent uses two equivalents of mercaptans, and the formation of one sulfone equivalent uses four equivalents of mercaptans.

The enzymatic cascade is operational until exhaustion of the substrates, reactants or cofactors or else by inhibition, inactivation or destruction of the enzymes E and/or D.

This cascade enables coproduction of sulfoxide or sulfone and disulfide, while regenerating the cofactor and the organic compound that are used.

It should be noted that the cascade may very well be commenced in the opposite direction: all the reactions described above may be reversed.

In this way one of the advantages of the invention is understood: it can be inserted into an overall process for deriving value from by-products resulting from the production of mercaptans and more particularly of methyl mercaptan. In the production of mercaptans, sulfides are by-products, as indicated above. By virtue of the invention, these sulfides are oxidized, preferably selectively, to sulfoxides or sulfones, and the mercaptans produced may in part be used to regenerate the cofactors used in the sulfoxidation, while themselves being converted to disulfides, which are other products of interest.

The present invention hence relates to a process for coproducing a disulfide and a sulfoxide or a sulfone, comprising the following steps:
a) preparing a composition M comprising:
   1) a sulfide,
   2) optionally an oxidizing agent,
   3) an organic compound bearing at least one thiol group,
   4) an enzyme E catalyzing the oxidation of said sulfide to sulfoxide or to sulfone,
   5) an enzyme D catalyzing the formation of a disulfide bridge between two equivalents of said organic compound bearing at least one thiol group to form a dimer, and
   6) a cofactor common to the two enzymes E and D;
b) conducting, preferably simultaneously, enzymatic reactions of sulfide oxidation and disulfide bridge formation, so as to obtain:
   a sulfoxide or a sulfone; and
   a dimer (of said organic compound);
c) reducing said dimer obtained in step b) by reaction, especially by chemical reaction, with a mercaptan, so as to obtain:
   the corresponding disulfide (corresponding to the mercaptan); and
   said organic compound bearing at least one thiol group; and
d) optionally recovering:
   the disulfide obtained in step c); and/or
   the sulfoxide or the sulfone obtained in step b);
   it being possible for said mercaptan to be added in any one of steps a), b) or c), and preferably the mercaptan is added in step a).

### BRIEF DESCRIPTION OF THE DRAWINGS

FIG. 1 illustrates the concentrations (in mM) of diethyl sulfide (DES), diethyl sulfoxide (DESO) and bis(2-hydroxyethyl) disulfide (Disulfide) present in the reaction medium as a function of the time (expressed in hours), when the oxidation reaction is catalyzed by the enzyme CHMO.

FIG. 2 illustrates the concentrations (in mM) of diethyl sulfoxide (DESO), diethyl sulfone (DESO₂) and bis(2-hydroxyethyl) disulfide (Disulfide) present in the reaction medium as a function of the time (expressed in hours), when the oxidation reaction is catalyzed by the enzyme CHMO.

FIG. 3 illustrates the concentration (in mM) of diethyl sulfoxide (DESO) present in the reaction medium as a function of the time (expressed in hours), when the reaction is catalyzed by the enzyme CHMO.

FIG. 4 illustrates an enzymatic cascade as according to an embodiment of the invention.

### DETAILED DESCRIPTION OF THE INVENTION

### Definitions

The term “(C₁-C₂₀)alkyl” denotes saturated aliphatic hydrocarbons which may be linear or branched and which comprise from 1 to 20 carbon atoms. Preferably the alkyls comprise from 1 to 12 carbon atoms, or even from 1 to 4 carbon atoms. Examples include methyl, ethyl, n-propyl, isopropyl, n-butyl, isobutyl, sec-butyl and tert-butyl. The term “branched” is understood to mean that an alkyl group is substituted along the main alkyl chain.

The term “(C₂-C₂o)alkenyl” denotes an alkyl as defined above that comprises at least one carbon-carbon double bond.

The term “(C₂-C₂o)alkynyl” denotes an alkyl as defined above that comprises at least one carbon-carbon triple bond.

The term “(C₆-C₁₀)aryl” denotes monocyclic, bicyclic or tricyclic aromatic hydrocarbon compounds, more particularly phenyl and naphthyl.

The term “(C₃-C₁₀)cycloalkyl” denotes monocyclic or bicyclic saturated aliphatic hydrocarbons comprising from 3 to 10 carbon atoms, such as cyclopropyl, cyclobutyl, cyclopentyl or cyclohexyl.

“(C₃-Cio)heterocycloalkane” is understood to be a cycloalkane comprising from 3 to 10 carbon atoms and comprising at least one sulfur atom, preferably tetrahydrothiophene, and optionally at least one other heteroatom.

“(C₄-C₁₀)heteroarene” is understood to be an arene comprising between 4 and 10 carbon atoms and comprising at least one sulfur atom, for example thiophene, and optionally at least one other heteroatom.

A heteroatom is understood in particular to be an atom selected from O, N, S, Si, P and halogens.

“Catalyst” is understood generally to be a substance which accelerates a reaction and which is unchanged at the end of this reaction. According to one embodiment, said enzyme E catalyzes the oxidation reaction of sulfides to sulfoxides or to sulfones. According to one embodiment, said enzyme D catalyzes the formation of a disulfide bridge between two equivalents of said organic compound bearing at least one thiol group to form a dimer.

A “catalytic amount” is understood in particular to be an amount sufficient to catalyze a reaction, more particularly to catalyze the oxidation of sulfides to sulfoxides or to sulfones and/or to form a disulfide bridge. More particularly, a reactant used in a catalytic amount is used in a smaller amount, for example between around 0.01% and 20% by weight, relative to the amount by weight of a reactant used in stoichiometric proportion.

The selectivity of a reaction generally represents the number of moles of product formed relative to the number of moles of reactant consumed following the reaction.

The usual definitions of conversion, of selectivity and of yield are as follows:

$$\begin{matrix}\text{Conversion} \text{=} \left(\text{number} \text{of} \text{moles} \text{of} \text{reactant} \text{in} \text{the} \text{initial} \text{state} \text{-} \text{number} \text{of} \text{moles} \text{of}\right) \\ \text{reactant} \text{remaining} \text{after} \text{the} \left(\text{reaction}\right) \text{/} \left(\text{number} \text{of} \text{moles} \text{of} \text{reactant} \text{in} \text{the} \text{initial}\right) \\ \left(\text{state}\right) \\ \text{Selectivity} \text{=} \text{} \text{number} \text{of} \text{moles} \text{of} \text{reactant} \text{converted} \text{into} \text{the} \text{desired} \text{product} \text{/} \left(\text{number}\right) \\ \text{of} \text{moles} \text{of} \text{reactant} \text{in} \text{the} \text{initial} \text{state} - \text{number} \text{of} \text{moles} \text{of} \text{reactant} \text{remaining} \text{after} \\ \text{the} \left(\text{reaction}\right) \\ \text{Yield} \text{=} \text{conversion} \times \text{selectivity}\end{matrix}$$

Hence “selective process for preparing sulfoxides” is understood especially to be a process which consumes sulfides and produces sulfoxides, without sulfones being formed (or with a negligible amount of sulfones being formed). According to one embodiment, the oxidation reaction of the sulfides to sulfoxides is chemoselective.

“Selective process for preparing sulfones” is understood especially to be a process which consumes sulfides and produces sulfones, without sulfoxides being formed (or with a negligible amount of sulfoxides being formed). According to one embodiment, the oxidation reaction of the sulfides to sulfones is chemoselective.

For example, the process according to the invention, more particularly step b), provides a selectivity of between 95% and 100%, preferably between 99% and 100%, for the sulfoxides or the sulfones.

The formation of a disulfide bridge between two equivalents of an organic compound bearing at least one thiol group to form a dimer is considered in particular to correspond to the formation of a disulfide bridge between two molecules of said organic compound (i.e., 2 R-SH give R-S-S-R).

### Process for Coproducing a Disulfide and a Sulfoxide or a Sulfone

The present invention more particularly relates to a process for coproducing a disulfide and a sulfoxide or a sulfone, comprising the following steps:
a) preparing a composition M comprising:
   1) a sulfide of formula R₁-S-R₂,
   2) optionally an oxidizing agent,
   3) an organic compound bearing at least one thiol group,
   4) an enzyme E catalyzing the oxidation of said sulfide to sulfoxide or to sulfone,
   5) an enzyme D catalyzing the formation of a disulfide bridge between two equivalents of said organic compound bearing at least one thiol group to form a dimer, and
   6) a cofactor common to the two enzymes E and D;
b) conducting, preferably simultaneously, enzymatic reactions of sulfide oxidation and disulfide bridge formation, so as to obtain:
   a sulfoxide or a sulfone of formula R₁-S(O)ₙ-R₂, with n = 1 or 2; and
   a dimer;
c) reducing the dimer obtained in step b) by reaction with a mercaptan of formula R₃-SH, so as to obtain:
   a disulfide of formula R₃-S-S-R₃; and
   said organic compound bearing at least one thiol group; and
d) optionally recovering:
   the disulfide obtained in step c); and/or
   the sulfoxide or the sulfone obtained in step b);
   it being possible for said mercaptan to be added in any one of steps a), b) or c), and preferably the mercaptan is added in step a);
   with R₁, R₂ and R₃ as defined hereinafter.

According to a preferred embodiment:
the sulfide is dimethyl sulfide;
the organic compound bearing at least one thiol group is glutathione;
the enzyme E is a Baeyer-Villiger Monooxygenase (BVMO), preferably a Cyclohexanone Monooxygenase (CHMO);
the enzyme D is a glutathione reductase; and
the cofactor common to the two enzymes E and D is NADP.

### Producing a Sulfoxide or a Sulfone

The process according to the invention comprises especially a step of conducting the enzymatic oxidation reaction of the sulfide to sulfoxide or to sulfone, said reaction being preferably selective.

In a first embodiment, when the sulfide is not wholly consumed during the step of conducting the enzymatic sulfide oxidation reaction, then the sulfoxide is selectively obtained. Hence, according to one embodiment, said composition M still comprises an amount of sulfide sufficient for the enzyme E to convert the sulfide to sulfoxide, preferably without formation of sulfone. According to one embodiment, the sulfide is supplied in excess in the composition M.

In this case, the amount of sulfide remaining after step b) of conducting the enzymatic reaction E may be between 0.0001% and 99.9% by weight, preferably between 0.1% and 99% by weight, preferably between 1% and 50% by weight, for example between 1% and 10% by weight, relative to the starting amount of sulfide by weight, that is to say of step a).

In a second embodiment, when the sulfide is wholly consumed during the step of conducting the enzymatic sulfide oxidation reaction, then the sulfone is selectively obtained. Hence the step of conducting the enzymatic oxidation reaction may in particular comprise the following two steps:
b1) complete oxidation of the sulfide to sulfoxide;
b2) oxidation of the sulfoxide to sulfone.

“Complete oxidation of the sulfide” means that the sulfide is consumed entirely during step b1). According to one embodiment, the sulfide is the limiting reactant (i.e., the reactant present in deficit) in the composition M. By “consumed entirely” is meant in particular that the amount of sulfide remaining after step b) of conducting the enzymatic reaction may be between 0% and 20% by weight, preferably between 0% and 5% by weight, for example between 0% and 1% by weight, and more preferably still between 0% and 0.01% by weight, relative to the starting amount of sulfide by weight, that is to say of step a).

This reaction is described in patent applications FR1906488 for the formation of sulfoxides and FR1906489 for the formation of sulfones.

### Coproducing a Disulfide and a Sulfoxide or a Sulfone

Steps b) and c) or steps a), b) and c) are preferably carried out in one and the same reactor; more preferably, steps b) and c) proceed simultaneously.

In step a), the various components of the composition M may be added in any order, for example by simple mixing of the various components in whichever order. The composition M may be prepared prior to introduction into the reactor or directly in the reactor (where step b) and optionally step c) proceed).

In step b), the enzymatic sulfide oxidation reaction may be carried out before or at the same time as the enzymatic disulfide bridge formation reaction.

More particularly, in step b), the dimer of the organic compound obtained is in oxidized form, whereas, in step c), the organic compound is in reduced form.

Step b) may especially subdivide into a plurality of steps as follows:
i) conducting the enzymatic sulfide oxidation reaction with the enzyme E, so as to give:
   a sulfoxide or a sulfone; and
   the cofactor common to the enzymes E and D in oxidized form;
ii) conducting the enzymatic disulfide bridge formation reaction with the enzyme D, so as to give:
   the cofactor common to the enzymes E and D in reduced form; and
   a dimer of said organic compound.

Step i) may be carried out before or at the same time as step ii).

Step b) of conducting the enzymatic reactions may be carried out at a pH of between 4 and 10, preferably between 6 and 8 and more preferably still between 7 and 8, for example 7.

Step b) of conducting the enzymatic reactions may be carried out at a temperature of between 5° C. and 100° C., preferably between 20° C. and 80° C. and more preferably still between 25° C. and 40° C.

The cells as defined below may be used in step b) directly in the absence of any treatment.

Step c) may be carried out under the same conditions, more particularly at the same pH and at the same temperature, as step b).

The pressure used for said enzymatic reaction and/or step c) may range from a reduced pressure relative to atmospheric pressure to several bar (several hundred kPa), depending on the reactants used and the equipment used.

According to one embodiment, the process according to the invention comprises a step b′), between step b) and step c), in which the enzymatic reactions are halted by inactivation of the enzyme and/or enzymes E and/or D. This step b′) may be carried out by known means such as heat shock (for example with a temperature of around 100° C.) or osmotic shock, application of a high pressure, addition of a solvent enabling destruction and/or precipitation of the cells and/or of the enzymes E and/or D, or pH modification (either a low pH of around 2 or a high pH of around 10).

The sulfide and/or the mercaptan and/or where appropriate said oxidizing agent may be added continuously, preferably in step a).

In step d), the sulfoxide or the sulfone and/or the disulfide may be recovered in liquid or solid form. The sulfoxide or the sulfone and/or the disulfide may be recovered in aqueous solution, in liquid form by decanting, or even in solid form by precipitation, depending on their solubility. The disulfide may be extracted in an organic phase or separated from the reaction mixture by techniques well known to those skilled in the art, for example by distillation after ultrafiltration or centrifugation.

Thereafter the products obtained may optionally be purified by conventional methods. For example, after separation of the cells (containing the enzymes E and D) by ultrafiltration or centrifugation, distillation may enable separation of the sulfoxide or the sulfone and the disulfide. This distillation may take place at atmospheric pressure, reduced pressure (for example under vacuum) or under higher pressure if those skilled in the art deem it to hold any advantage. Membrane separation may also be contemplated for the purpose of reducing the water content of the mixture for distillation, or of accelerating a crystallization process. If the sulfoxide or the sulfone and/or the sulfide has been recovered by decanting from an aqueous reaction mixture, drying over molecular sieve (or any other drying method) may be contemplated.

Said process may be carried out batchwise or continuously. The advantages procured by the process of the invention are many. These advantages include the possibility of working in aqueous solution, under very mild temperature and pressure conditions and under pH conditions close to neutrality. All of these conditions are typical of a biocatalytic process referred to as being “green” or “sustainable”.

The composition M may comprise the organic compound, the enzyme E, the enzyme D and the cofactor in catalytic amount. The composition M may comprise:
1) a stoichiometric amount of a sulfide,
2) a stoichiometric amount of an oxidizing agent,
3) a catalytic amount of an organic compound bearing at least one thiol group,
4) a catalytic amount of an enzyme E catalyzing the oxidation of said sulfide to sulfoxide or to sulfone,
5) a catalytic amount of an enzyme D catalyzing the formation of a disulfide bridge between two equivalents of said organic compound bearing at least one thiol group to form a dimer,
6) a catalytic amount of a cofactor common to the two enzymes E and D; and
7) a stoichiometric amount of a mercaptan.

More particularly, the mercaptan/sulfide molar ratio is between 0.1 and 100, more preferably between 1 and 5 and preferably between 2 and 4, for example 2.

### Sulfide

A sulfide is in particular an organic sulfide, this being any organic compound comprising at least one -C-S-C- function.

According to one embodiment, the composition M comprises at least one sulfide. It may for example comprise one, two or multiple different sulfides.

Said sulfide may be symmetrical, meaning that the sulfur atom represents a center of symmetry relative to the compound.

According to one embodiment, said sulfide has the following general formula:

$${\text{R}}_{\text{1}} {\text{-S-R}}_{\text{2}}$$

in which,
R₁ and R₂ may be identical or different and are selected, independently of one another, from the group consisting of:
(C₁-C₂o)alkyl, (C₂-C₂₀)alkenyl, (C₂-C₂₀)alkynyl, (C₃-C₁₀)cycloalkyl and
(C₆-C₁₀)aryl or
R₁ and R₂ form a ring with the sulfur atom to which they are attached, preferably a (C₃-C₁₀)heterocycloalkane or (C₄-C₁₀)heteroarene group;
it being possible for said alkyl, alkenyl, alkynyl, cycloalkyl, aryl, heterocycloalkane and heteroarene groups optionally to be substituted by one or more substituents;
and it being possible for said alkyl, alkenyl, alkynyl, cycloalkyl and aryl groups to comprise one or more heteroatoms.

Said alkyl, alkenyl, alkynyl, cycloalkyl, aryl, heterocycloalkane and heteroarene groups may optionally be substituted by one or more substituents selected from the group consisting of:
(C₁-C₂o)alkyl, (C₃-C₁₀)cycloalkyl and (C₆-C₁₀)aryl;
and may be optionally functionalized with one or more functions selected, without limitation and by way of example, from alcohol, aldehyde, ketone, acid, amide, nitrile and ester functions or else functions bearing sulfur, phosphorus and silicon.

According to one embodiment, said alkyl, alkenyl, alkynyl, cycloalkyl, aryl, heterocycloalkane and heteroarene groups may optionally be substituted by one or more substituents selected from the group consisting of: (C₁-C₂o)alkyl, (C₃-C₁₀)cycloalkyl, (C₆-C₁₀)aryl, -OH, -C(O)OH, -C(O)H, -C(O)-NH₂, -NH₂, -NHR, -NRR′, -C(O)-, -C(O)-NHR′, -C(O)-NRR′, -COOR and -CN; in which R and R′ represent, independently of one another, a (C₁-C₂o)alkyl group.

According to one preferred embodiment, R₁ and R₂ may be identical or different and are selected, independently of one another, from the group consisting of:
(C₁-C₂o)alkyl, (C₂-C₂o)alkenyl, (C₂-C₂₀)alkynyl and (C₃-C₁₀)cycloalkyl or
R₁ and R₂ form, together with the sulfur atom to which they are attached, a (C₃-C₁₀)heterocycloalkane group.

R₁ and R₂ are preferably selected from (C₁-C₂₀)alkyls or R₁ and R₂ form, together with the sulfur atom bearing them, a (C3-C₁₀)heterocycloalkane. The radicals R₁ and R₂ of said sulfide are preferably identical (i.e., so forming a symmetrical sulfide).

The sulfide is more preferably selected from dimethyl sulfide, diethyl sulfide, dipropyl sulfide, dibutyl sulfide, dioctyl sulfide, didodecyl sulfide and tetrahydrothiophene. The sulfide may be selected from dimethyl sulfide, diethyl sulfide, di-n-propyl sulfide, diisopropyl sulfide, di-n-butyl sulfide, diisobutyl sulfide, di-sec-butyl sulfide, di-tert-butyl sulfide, di-n-octyl sulfide, di-n-dodecyl sulfide and tetrahydrothiophene. Dimethyl sulfide is particularly preferred according to the invention. According to one embodiment, the sulfide is symmetrical.

### Mercaptan

By mercaptan is meant in particular an organic mercaptan, i.e. any organic compound comprising at least one function of -C-SH type.

According to one embodiment, the mercaptan is of general formula R₃-SH, in which R₃ is an optionally substituted, saturated, linear, branched or cyclic hydrocarbon radical.

More particularly, the mercaptan is of general formula R₃-SH, in which R₃ is a saturated, linear, branched or cyclic hydrocarbon radical optionally substituted by at least one group selected from the group consisting of:
-OH, -C(O)OH, -NH₂, -ORₐ, -C(O)ORₐ, -N(Rₐ)H, -NRₐR_{b} and (C₆-C₁₀)aryl;
with Rₐ and R_{b} being selected, independently of one another, is from (C₁-C₂₀)alkyls.

The mercaptan is preferably of general formula R₃-SH, in which R₃ is a saturated, linear, branched or cyclic hydrocarbon radical optionally substituted by at least one, for example one or two, group(s) selected from the group consisting of: -OH, -C(O)OH, -NH₂ and (C₆-C₁₀)aryl, more preferably -OH, -C(O)OH and -NH₂.

R₃ is more particularly selected from the group consisting of methyl, ethyl, octyl and dodecyl, and preferably R₃ is a methyl.

The mercaptan may be selected from the group consisting of: mercaptoethanol, methyl mercaptan, ethyl mercaptan, propyl mercaptan, butyl mercaptan, octyl mercaptan, dodecyl mercaptan, benzyl mercaptan, thioglycolic acid, 3-mercaptopropionic acid, cysteine and homocysteine.

The mercaptan is especially selected from the group consisting of: mercaptoethanol, methyl mercaptan, ethyl mercaptan, n-propyl mercaptan, isopropyl mercaptan (or 2-propanethiol), n-butyl mercaptan, sec-butyl mercaptan, tert-butyl mercaptan, n-octyl mercaptan, tert-octyl mercaptan, n-dodecyl mercaptan, tert-dodecyl mercaptan, benzyl mercaptan, thioglycolic acid, 3-mercaptopropionic acid, cysteine and homocysteine.

The mercaptan is preferably methyl mercaptan.

### Oxidizing Agent

Oxidizing agent is understood to be any compound that is able to oxidize a sulfide to sulfoxide or to sulfone.

The oxidizing agent may be selected from the group consisting of air, oxygen-depleted air, oxygen-enriched air and pure oxygen.

When air (air which may be depleted or enriched in oxygen) is used, it is obviously the oxygen within the air that is consumed during the enzymatic oxidation reaction conducted in step b) as oxidizing agent.

When the oxidizing agent is in gaseous form, it is present in the composition M in the form of a dissolved gas. The percentage of oxygen in the enriched or depleted air is selected according to the reaction rate and to the compatibility with the enzymatic system in a manner known to those skilled in the art.

The oxidizing agent may be in a stoichiometric amount or in excess in the composition M. In excess, the sulfide present is consumed entirely with the oxidizing agent in the enzymatic sulfide oxidation reaction conducted in step b) (formation of sulfone).

The oxidizing agent may be in a substoichiometric amount in the composition M. Thus, the sulfide present is consumed partly with the oxidizing agent in the enzymatic sulfide oxidation reaction conducted in step b) but not entirely (formation of sulfoxide).

At the end of said oxidation reaction, the oxygen is generally converted to water when the enzyme E used is a monooxygenase, or consumed entirely when the enzyme E is a dioxygenase. The process according to the invention is therefore particularly advantageous in terms of emissions and of environmental friendliness.

### Enzyme E

“Oxidizing enzyme” is understood in particular to be the enzyme E, i.e. an enzyme enabling the oxidation of sulfides to sulfoxides and/or sulfones and requiring the use of a cofactor.

Said enzyme E may be an oxidoreductase, preferably an oxidoreductase selected from the group consisting of monooxygenases and dioxygenases, more preferably still from monooxygenases.

Said enzyme E is preferably a Baeyer-Villiger monooxygenase (BVMO).

More preferably still, and among the BVMOs, the enzyme E may be a cyclohexanone monooxygenase (CHMO) and more particularly a cyclohexanone 1,2-monooxygenase, a cyclopentanone monooxygenase (CPMO) and more particularly a cyclopentanone 1,2-monooxygenase, or a hydroxyacetophenone monooxygenase (HAPMO) and more particularly a 4-hydroxyacetophenone monooxygenase.

The cyclohexanone 1,2-monooxygenases are in particular from class EC 1.14.13.22.

According to one particular embodiment, the CHMO is a CHMO from Acinetobacter sp. (for example, of strain NCIMB 9871) and/or a CHMO encoded by the gene chnB belonging to cluster AB006902.

The cyclopentanone 1,2-monooxygenases are in particular from class EC 1.14.13.16.

According to one particular embodiment, the CPMO is a CPMO from Comamonas sp. (for example, the strain NCIMB 9872) and/or a CPMO encoded by the gene cpnB.

The hydroxyacetophenone monooxygenases are in particular from class EC 1.14.13.84. According to one particular embodiment, the HAPMO is a HAPMO from Pseudomonas fluorescens that is encoded by the gene hapE.

### Enzyme D and Organic Compound Bearing a Thiol Group

Organic compound bearing a thiol group is understood to be any hydrocarbon compound with or without heteroatoms and/or any type of known chemical function that bears at least one -SH group (hereinafter organic compound). The organic compound according to the invention may comprise one or two thiol groups (-SH groups). This compound may exist in dimer form, the dimer being formed owing to a disulfide bridge between two equivalents of said organic compound (2 R-SH give R-S-S-R).

The organic compound may be selected from the group consisting of an amino acid bearing a thiol group, a peptide bearing a thiol group, mycothiol (CAS No. 192126-76-4) and dihydrolipoic acid (CAS No. 462-20-4). Said organic compound is preferably an amino acid bearing a thiol group or a peptide bearing a thiol group. Said organic compound is selected more particularly from the group consisting of: cysteine, homocysteine, glutathione, thioredoxin, mycothiol and dihydrolipoic acid.

According to one embodiment, when the mercaptan according to the invention is cysteine or homocysteine, then said organic compound is different from cysteine or homocysteine (respectively). According to one embodiment, the organic compound and the mercaptan are different.

Said organic compound is selected more specifically from the group consisting of: cysteine, homocysteine, glutathione and thioredoxin.

Particularly preferably, said organic compound is glutathione. The glutathione (GSH)/glutathione disulfide (GSSG) couple is widely known in biology. In reduced form (glutathione) or oxidized form (glutathione disulfide), this entity forms an important redox couple in cells.

The enzyme D catalyzes the formation of a disulfide bridge between two equivalents of said organic compound to form a dimer (called a, one or the dimer in the description). In particular, it catalyzes the formation of a disulfide bridge between two equivalents of an amino acid bearing a thiol group or between two equivalents of a peptide bearing a thiol group to form a diamino acid or a dipeptide. The enzyme D may be defined as a “recycling enzyme” for the reduced or oxidized common cofactor, and preferably it recycles the oxidized common cofactor to reduced common cofactor.

It may be a reductase or a dehydrogenase, and preferably it may be selected from the group consisting of glutathione reductase, thioredoxin reductase, cysteine reductase, homocysteine reductase, mycothiol disulfide reductase and dihydrolipoyl dehydrogenase. The enzyme D is selected more particularly from the group consisting of glutathione reductase, thioredoxin reductase, cysteine reductase and homocysteine reductase.

The glutathione reductase may be represented by the enzymatic classification numbers EC 1.8.1.7 or EC 1.6.4.2; the cysteine reductase by the number EC 1.8.1.6; the thioredoxin reductase by the numbers EC 1.8.1.9 or EC 1.6.4.5; the mycothione reductase by the number EC 1.8.1.15 (this enzyme is also called mycothiol disulfide reductase). Particularly preferably, said enzyme D is glutathione reductase.

More specifically, each organic compound is coupled with its corresponding enzyme D, thus enabling the corresponding dimer to be formed. The organic compound/enzyme D enzymatic couples or complexes below are therefore particularly useful for implementing the process according to the invention:
glutathione/glutathione reductase,
thioredoxin/thioredoxin reductase,
cysteine/cysteine reductase,
homocysteine/homocysteine reductase,
mycothiol/mycothiol disulfide reductase, and
dihydrolipoic acid/dihydrolipoyl dehydrogenase.

### Common Cofactor(s)

“Common cofactor” is understood especially to be a cofactor needed for the catalytic activity of the enzyme E and of the enzyme D as they are defined above and/or allowing their catalytic activity to be enhanced. “Cofactor common” to the enzymes E and D is understood preferably to be a cofactor capable of being reduced and/or oxidized by the action of these enzymes.

According to one embodiment, one or two cofactors or more are present in the composition M. For example, it is possible to admix the composition M with a cofactor already present naturally in the enzyme E and/or in the enzyme D, in addition to another cofactor.

Said cofactor may be selected from nicotine cofactors and flavin cofactors. More particularly, said cofactor may be selected from the group consisting of: nicotinamide adenine dinucleotide (NAD), nicotinamide adenine dinucleotide phosphate (NADP), flavin mononucleotide (FMN), flavin adenine dinucleotide (FAD) and/or the corresponding reduced form thereof (namely, NADH,H+, NADPH,H+, FMNH₂, FADH₂).

The cofactors listed above are advantageously used in their reduced forms (for example, NADPH, H+) and/or their oxidized forms (for example, NADP+), that is to say that they can be added in these reduced and/or oxidized forms to the composition M, preferably in reduced form.

Preferably, the enzyme E used is cyclohexanone monooxygenase, for example the cyclohexanone monooxygenase from Acinetobacter sp., and the cofactor used is NADP, optionally supplemented by FAD, and the enzyme D is glutathione reductase.

The composition M according to the invention may also comprise:
optionally one or more solvents chosen from water, buffers such as phosphate buffers, Tris-HCI, Tris base, ammonium bicarbonate, ammonium acetate, HEPES (4-(2-hydroxyethyl)-1-piperazineethanesulfonic acid), CHES (N-cyclohexyl-2-aminoethanesulfonic acid), or salts such as sodium chloride, potassium chloride, or mixtures thereof;
optionally additives such as surfactants, in order in particular to promote the solubility of one or more reactants or substrates of the enzymatic reaction.

Preferably, the composition M is an aqueous solution. For example, said composition M comprises between 50% and 99% by weight of water, preferably between 80% and 97% by weight of water, relative to the total weight of the composition M.

According to one embodiment, the composition M is deemed to comprise the reaction mixture.

The various components of the composition M prepared in step a) above are readily obtainable commercially or may be prepared by techniques well known to the skilled person. These different elements may be in solid, liquid or gaseous form and may very advantageously be rendered into solution or dissolved in water or any other solvent to be used in the process of the invention. The enzymes used may also be grafted onto a support (in the case of supported enzymes).

According to one embodiment, the enzymes E and/or D, optionally the organic compound and optionally the common cofactor are:
in isolated and/or purified form, for example, in aqueous solution;
or in a crude extract, i.e., in an extract of milled cells; or
in whole cells.

Whole cells are used with preference. The [sulfide] (in mmol/L)/[cells] (in g_{cdw}.L⁻¹) ratio may be between 0.01 and 10, preferably between 0.01 and 3, mmol/g_{cdw}, preferably during step b) of carrying out the enzymatic reaction. The concentration by mass in grams of dry cells (g_{CDW} for Cells Dry Weight) is determined by conventional techniques.

The enzymes E and/or D may or may not be overexpressed in said cells, which are referred to below as host cells.

The host cell may be any host appropriate for producing an enzyme E and/or D from the expression of the corresponding coding gene. This gene will then be either located in the genome of the host or carried by an expression vector such as those defined below.

For the purposes of the present invention, “host cell” is in particular understood to be a prokaryotic or eukaryotic cell. Host cells commonly used for the expression of recombinant or non-recombinant proteins include in particular cells of bacteria such as Escherichia coli or Bacillus sp., or Pseudomonas, cells of yeasts such as Saccharomyces cerevisiae or Pichia pastoris, cells of fungi such as Aspergillus niger, Penicillium funiculosum or Trichoderma reesei, insect cells such as Sf9 cells, or else mammalian (in particular, human) cells such as the HEK 293, PER-C6 or CHO cell lines.

Said host cells may be in stationary growth phase, for example having been removed from the culture medium.

Preferably, the enzymes E and/or D, optionally the organic compound and optionally the common cofactor are expressed in the bacterium Escherichia coli. The CHMO and/or the HAPMO is/are preferably expressed within a strain of Escherichia coli, such as, for example, Escherichia coli BL21(DE3).

### Integration of an Expression Vector Comprising the Coding Sequence for the Enzyme E and/or D in the Cellular Host

When an expression vector such as a plasmid is used, transformation of the prokaryotic and eukaryotic cells is a technique well known to the skilled person, as for example by lipofection, electroporation, heat shock, or by chemical methods. The expression vector and the method of introducing the expression vector within the host cell are chosen according to the host cell selected. This transformation step yields a transformed cell that expresses a gene coding for a recombinant enzyme E and/or D. The cell may be cultured, in a culturing/incubating step, to produce the enzyme E and/or D.

The incubation/culturing of prokaryotic and eukaryotic cells is a technique well known to the skilled person, who is able to determine, for example, the culture medium or else the temperature and time conditions. Depending on the vector used, an induction period -corresponding to increased production of the enzyme E and/or D - may be observed. Consideration may be given to using a weak (as for example arabinose for the vector pBad) or strong (as for example isopropyl β-D-1-thiogalactoside (IPTG) for the vectors pET22b, pRSF, etc.) inductor. Production of the enzyme E and/or D by the host cell may be verified using the technique of SDS-PAGE electrophoresis or the Western blot technique.

An “expression vector” is a DNA molecule of reduced size into which a nucleotide sequence of interest can be inserted. Selection may be made from a number of known expression vectors, such as plasmids, cosmids, phages, etc.

The vector is selected particularly as a function of the cellular host that is used.

The expression vector in question may be, for example, that described in document WO 83/004261.

### Integration of the Coding Sequence for the Enzyme E And/or D in the Genome of the Host Cell in the Absence of an Expression Vector

The nucleotide sequence coding for the enzyme E and/or D may be integrated into the genome of the host cell by any known method, such as, for example, by homologous recombination or else by the system CRISPR-Cas9, etc. Production of the enzyme E and/or D by the host cell may be verified using the technique of SDS-PAGE electrophoresis or the Western blot technique.

### Isolation And/or Purification of the Enzyme E And/or D for Use in Isolated And/or Purified Form

Following transformation and culturing/incubation of the transformed host cell, a step of isolation and optionally of purification of the enzyme E and/or D may be carried out. In this way, the process according to the invention is not carried out in the presence of the host cells but by the enzyme E and/or D in solution in the composition M, preferably in aqueous solution.

The isolation and/or the purification of said enzyme E and/or D produced may be carried out by any means known to those skilled in the art. This may for example involve a technique selected from electrophoresis, molecular sieving, ultracentrifugation, differential precipitation, for example with ammonium sulfate, ultrafiltration, membrane or gel filtration, ion exchange, separation via hydrophobic interactions, or affinity chromatography, such as IMAC, for example.

### Mode of Lysis of the Host Cell, Preparation of a Crude Extract of Milled Cells

The cell lysate may be obtained by various known techniques such as sonication, pressure (French press), via the use of chemical agents (e.g., Triton), etc. The lysate obtained corresponds to a crude extract of milled cells.

### Composition and Uses

The present invention also relates to a composition comprising:
1) a sulfide,
2) optionally an oxidizing agent,
3) an organic compound bearing at least one thiol group,
4) an enzyme E catalyzing the oxidation of said sulfide to sulfoxide or to sulfone,
5) an enzyme D catalyzing the formation of a disulfide bridge between two equivalents of said organic compound bearing at least one thiol group to form a dimer,
6) a cofactor common to the two enzymes E and D, and
7) a mercaptan.

The invention also relates to the use of a mercaptan for regenerating or recycling a cofactor (enzymatic cofactor, especially as defined above), preferably a cofactor used in the enzymatic oxidation of a sulfide. “Regenerating” or “recycling” is understood especially to be the passage from an oxidized form to a reduced form of the cofactor, or vice versa.

The present invention also relates to the use of a mercaptan for reducing a disulfide bridge formed between two equivalents of an organic compound bearing at least one thiol group, said mercaptan being preferably converted to the corresponding disulfide. Said disulfide bridge is preferably formed by enzymatic catalysis, especially as defined above for the enzyme D. Preferably, the mercaptan is methyl mercaptan and the organic compound is glutathione. This use of the mercaptan is envisaged especially as a pathway for regenerating or recycling a cofactor, especially a cofactor used in the enzymatic oxidation of a sulfide.

The invention also relates to a process for enzymatic oxidation of a sulfide, comprising a step of regenerating or recycling a cofactor by a mercaptan.

The present invention also relates to a process using a mercaptan for reducing a disulfide bridge formed between two equivalents of an organic compound bearing at least one thiol group, said mercaptan being preferably converted to the corresponding disulfide.

More particularly, the sulfide, the oxidizing agent, the organic compound, the enzyme E, the enzyme D, the cofactor, the mercaptan and the enzymatic and chemical reactions are as defined above, especially in the context of the coproduction process according to the invention.

### BRIEF DESCRIPTION OF THE DRAWINGS

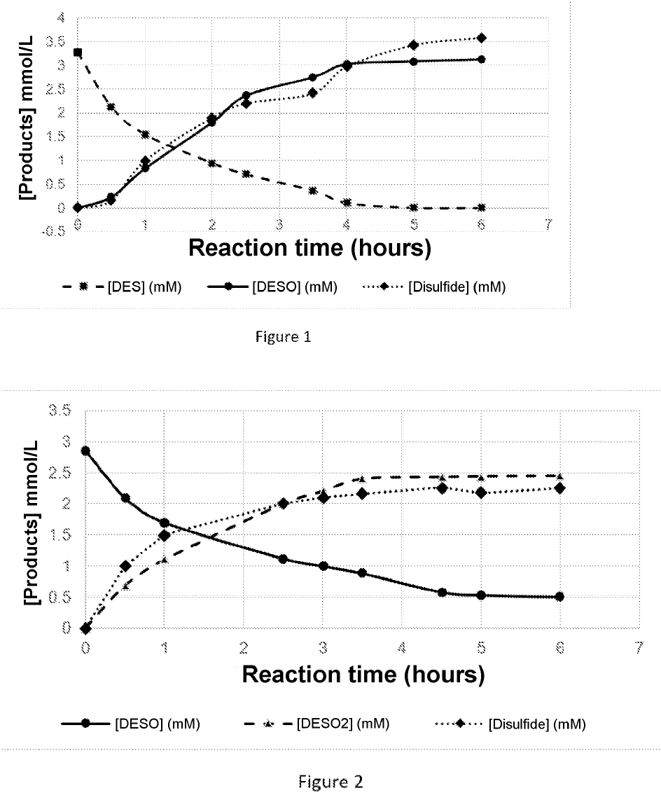

FIG. 1: FIG. 1 represents the concentrations (in mM) of diethyl sulfide (DES), diethyl sulfoxide (DESO) and bis(2-hydroxyethyl) disulfide (Disulfide) present in the reaction medium as a function of the time (expressed in hours), when the oxidation reaction is catalyzed by the enzyme CHMO. The elements of the cascade enabling the recycling of the cofactor NADP were also added: the cells overexpressing glutathione reductase (GR) from E. coli were added at the same concentration as the cells expressing CHMO, the oxidized glutathione (GSSG) was introduced in a catalytic amount and the 2-mercaptoethanol at twice the concentration relative to the DES.

FIG. 2: FIG. 2 represents the concentrations (in mM) of diethyl sulfoxide (DESO), diethyl sulfone (DESO₂) and bis(2-hydroxyethyl) disulfide (Disulfide) present in the reaction medium as a function of the time (expressed in hours), when the oxidation reaction is catalyzed by the enzyme CHMO. The elements of the cascade enabling the recycling of the cofactor NADP were also added: the cells overexpressing glutathione reductase (GR) from E. coli were added at the same concentration as the cells expressing CHMO, the oxidized glutathione (GSSG) was introduced in a catalytic amount and the 2-mercaptoethanol at twice the concentration relative to the DESO.

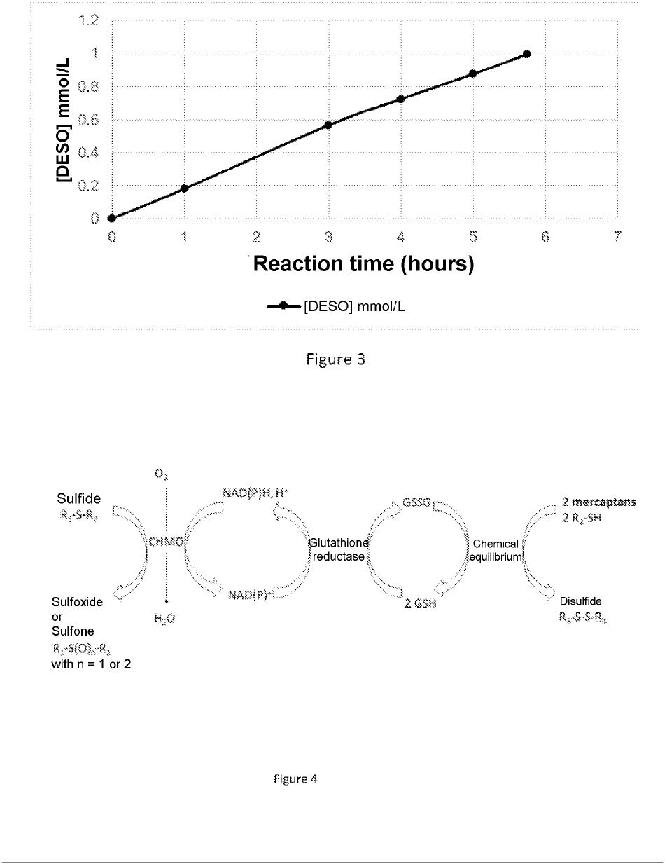

FIG. 3: FIG. 3 represents the concentration (in mM) of diethyl sulfoxide (DESO) present in the reaction medium as a function of the time (expressed in hours), when the reaction is catalyzed by the enzyme CHMO. In this example, 2-mercaptoethanol was replaced by methyl mercaptan (MeSH) as a proton donor. The elements of the cascade enabling the recycling of the cofactor NADP were also added: the cells overexpressing glutathione reductase (GR) from E. coli were added at the same concentration as the cells expressing CHMO, the oxidized glutathione (GSSG) was introduced in a catalytic amount and the MeSH is added gradually to the reaction medium at a flow rate of about 4 mmol/L/h.

FIG. 4: FIG. 4 is an illustration of an enzymatic cascade as according to the invention.

The mercaptan and the sulfide are introduced into a reaction medium comprising:
CHMO (corresponding to the enzyme E),
NADPH (common cofactor),
glutathione reductase (corresponding to the enzyme D),
glutathione disulfide or glutathione (corresponding to the dimer or to the organic compound comprising at least one thiol group).

In particular, air is introduced continuously enabling the oxidation of the sulfide to sulfoxide or to sulfone depending on the mode of introduction of the sulfide. The enzymatic cascade is then created.

The common cofactor, the NADPH, is oxidized to NADP+ concomitantly with the formation of sulfoxide or sulfone. The NADP+, owing to the presence of two molecules of glutathione, is reduced again to NADPH,H+, the 2 glutathione molecules (2 GSH) giving glutathione disulfide (GSSG). To terminate the cascade, the glutathione disulfide, via a chemical equilibrium, enables the oxidation of 2 molecules of mercaptans to the corresponding disulfide, thereby regenerating 2 molecules of glutathione.

The examples below are given for illustrative purposes and do not limit the present invention.

### EXAMPLES

### Example 1: Selective Synthesis of Diethyl Sulfoxide From Diethyl Sulfide via the Use of 2-Mercaptoethanol

### I. Preparation of the Biocatalyst

### Production of CycloHexanone MonoOxygenase (CHMO)

A strain of Escherichia coli BL21(DE3) (sold by Merck Millipore) expressing the chnB gene inserted into the plasmid pET22b (sold by Promega, Qiagen) was constructed beforehand. It enables the heterologous expression of CycloHexanone MonoOxygenase (CHMO) from Acinetobacter sp.

It is understood that said strain contains both CHMO and the cofactors of CHMO, namely NADP and FAD.

This strain was precultured and cultured by the techniques known to the skilled person.

After an induction phase triggered by adding isopropyl β-D-1-thiogalactoside (IPTG) at a final concentration of 0.85 mmol/L, a certain volume of the culture is centrifuged (10 min, 5000 g, 4° C.) to give the desired amount of cells.

### Production of Glutathione Reductase (GR)

A strain of Escherichia coli BL21(DE3) (sold by Merck Millipore) expressing the gor gene inserted onto the plasmid pET26b+ (sold by Promega, Qiagen) was designed according to the techniques known to those skilled in the art. It allows the heterologous expression of the glutathione reductase (GR) from Escherichia coli.

It is understood that said strain contains both GR and the cofactor of GR, namely NADP.

This strain was precultured and cultured by the techniques known to those skilled in the art.

After an induction phase triggered by adding isopropyl β-D-1-thiogalactoside (IPTG), a certain volume of the culture is centrifuged (10 min, 5000 g, 4° C.) so as to give the desired amount of cells.

### II. Bioconversion

In this example, the pellets of fresh cells are resuspended in 32 mL of a 0.1 mol/L phosphate buffer at pH 8. The cell concentration then obtained is 62 ODU/mL or else 20 g_{CDW}/L (where CDW stands for cells dry weight) for the cells overexpressing CHMO and GR.

In a 250 mL flask, initial concentrations of 3 mmol/L of diethyl sulfide (DES), 6 mmol/L of 2-mercaptoethanol and 0.25 mmol/L of oxidized glutathione (GSSG) are present at t=0 in a final volume of 32 mL.

At regular intervals, 200 µL of the reaction mixture are withdrawn and diluted in 1000 µL of an acetonitrile solution. After centrifugation (5 min, 12 500 g), the supernatant is injected in GC for quantitative measurement of the diethyl sulfoxide (DESO) and the bis(2-hydroxyethyl) disulfide (Disulfide) formed during the reaction, using a calibration standard range obtained beforehand. Under the conditions of the analysis performed, the minimum measurable concentration is 50 µM.

A linear increase in the amount of DESO is measured over time, with no detection of the sulfone (DESO₂). The initial rate of oxidation of the sulfide is then 1 mmol of DES oxidized per liter of medium per hour (FIG. 1). An analogous amount of bis(2-hydroxyethyl) disulfide is produced concomitantly with the production of DESO, thereby verifying the proper functioning of the coupled GSSG/GR system for recycling the cofactor. It should be noted that the concentration of bis(2-hydroxyethyl) disulfide as a function of the time was determined after subtraction of the background noise associated with the slow spontaneous oxidation of the 2-mercaptoethanol (background noise determined in the presence of all the elements of the cascade (under the same conditions) except for the cells overexpressing CHMO).

The reaction catalyzed by CHMO is chemoselective, since the reaction in which the DESO is converted into DESO₂ takes place only when the DES has been entirely consumed. Expressed alternatively, the sulfone does not form when there is DES in the reaction medium.

The selectivity obtained is around 100%. When the DES is still present in the reaction medium, the sulfone is not detected with the analytical tools used. At the end of the reaction (t = 4 h), around 100% of DESO is obtained, with no DESO₂ being detected by the analytical tools used.

### Example 2: Selective Synthesis of Diethyl Sulfone From Diethyl Sulfoxide via the Use of 2-Mercaptoethanol as Hydrogen Donor

### I. Preparation of the Biocatalyst

The same strains as those described in example 1 were used in this example, that is to say the strains overexpressing CHMO and GR from E. coli. Centrifugation is carried out (10 min, 5000 g, 4° C.) and the pellets are then resuspended in 32 mL of a 0.1 mol/L phosphate buffer, pH 8. A cell concentration of 62 ODU/mL (or around 20 g_{CDW}/L) of each strain is then used for the reaction test.

### II. Bioconversion

In a 250 mL flask, 3 mmol/L of diethyl sulfoxide (DESO), 6 mmol/L of 2-mercaptoethanol and 0.25 mmol/L of oxidized glutathione (GSSG) are added simultaneously at t=0 in a final volume of 32 mL.

At regular intervals, 200 µL of the reaction mixture are withdrawn and diluted in 1000 µL of an acetonitrile solution. After centrifugation (5 min, 12 500 g), the supernatant is injected in GC for quantitative measurement of the diethyl sulfone (DESO₂) and the bis(2-hydroxyethyl) disulfide (Disulfide) formed during the reaction, using a calibration standard range obtained previously.

A linear increase in the amount of DESO₂ is measured over time. The initial rate of oxidation of the sulfoxide is then 0.75 mmol of DESO oxidized per liter of medium per hour (FIG. 2). In parallel, an analogous amount of bis(2-hydroxyethyl) disulfide is produced concomitantly with the production of DESO₂. Consequently, the added 2-mercaptoethanol does indeed make it possible to recycle the cofactor NADP via a cascade-dependent pathway. The background noise was deducted in the same manner as in example 1.

### Example 3: Selective Synthesis of Diethyl Sulfoxide From Diethyl Sulfide via the Use of Methyl Mercaptan (MeSH) as Hydrogen Donor

### I. Preparation of the Biocatalyst

The same strains as those described in example 1 and 2 were used in this example, that is to say the strains overexpressing CHMO and GR from E. coli. The 2-mercaptoethanol was replaced by MeSH in this biocatalyzed reaction.

### II. Bioconversion

In this example, the pellets of fresh cells are resuspended in 200 mL of a 0.1 mol/L phosphate buffer at pH 8. The cell concentration then obtained is 62 ODU/mL or else 20 g_{CDW}/L (where CDW stands for cells dry weight) for the cells overexpressing CHMO and GR.

A 1 L reactor is charged simultaneously at t = 0 with: 10 mmol/L of diethyl sulfide (DES), 0.25 mmol/L of oxidized glutathione (GSSG) and also the enzymatic solution prepared beforehand. Additionally, methyl mercaptan is added gradually to the reaction medium by sulfuric acid acidification of sodium methyl mercaptan (flow rate adjusted to around 4 mmol/L/h). Then pure diethyl sulfide is added gradually at a flow rate of 40 µL/h to ensure a constant concentration of DES and a bladder enables the regular addition of O₂ which is needed for carrying out the reaction. The reaction is initiated with stirring at a controlled temperature of 30° C.

At regular intervals, 200 µL of the reaction mixture are withdrawn and diluted in 1000 µL of an acetonitrile solution. After centrifugation (5 min, 12 500 g), the supernatant is injected in GC for quantitative measurement of the diethyl sulfoxide (DESO) and the diethyl sulfone (DESO₂) potentially formed during the reaction, using a calibration standard range obtained previously. Under the conditions of the analysis performed, the minimum measurable concentration is 50 µM.

A linear increase in the amount of DESO is measured over time, with no detection of the sulfone (DESO₂). The initial rate of oxidation of the sulfide is then 0.2 mmol of DES oxidized per liter of medium per hour (FIG. 3). Dimethyl disulfide (DMDS) is produced concomitantly with the production of DESO, thereby verifying the proper functioning of the coupled GSSG/GR system for recycling the cofactor.

The reaction catalyzed by CHMO is chemoselective, because the reaction in which the DESO is converted into DESO₂ does not take place since DES is present in the reaction medium throughout the reaction.

1. A process for coproducing a disulfide and a sulfoxide or a sulfone, comprising :
a) preparing a composition M comprising:
1) a sulfide,
2) optionally an oxidizing agent,
3) an organic compound bearing at least one thiol group,
4) an enzyme E catalyzing oxidation of said sulfide to sulfoxide or to sulfone,
5) an enzyme D catalyzing formation of a disulfide bridge between two equivalents of said organic compound bearing at least one thiol group to form a dimer, and
6) a cofactor common to the two enzymes E and D;
b) conducting enzymatic reactions of sulfide oxidation and disulfide bridge formation, so as to obtain:
a sulfoxide or a sulfone; and
a dimer;
c) reducing the dimer obtained in step b) by reaction with a mercaptan, so as to obtain:
the corresponding disulfide; and
said organic compound bearing at least one thiol group; and
d) optionally recovering:
the disulfide obtained in step c); and/or
the sulfoxide or the sulfone obtained in step b); it being possible for said mercaptan to be added in any one of steps a), b) or c).

2. The coproduction process as claimed in claim 1, wherein the mercaptan is of general formula R₃-SH, in which R₃ is an optionally substituted, saturated, linear, branched or cyclic hydrocarbon radical.

3. The coproduction process as claimed in claim 2, wherein R₃ is selected from the group consisting of methyl, ethyl, octyl and dodecyl methyl.

4. The coproduction process as claimed in claim 2, wherein the mercaptan is selected from the group consisting of: mercaptoethanol, methyl mercaptan, ethyl mercaptan, n-propyl mercaptan, isopropyl mercaptan (or 2-propanethiol), n-butyl mercaptan, sec-butyl mercaptan, tert-butyl mercaptan, n-octyl mercaptan, tert-octyl mercaptan, n-dodecyl mercaptan, tert-dodecyl mercaptan, benzyl mercaptan, thioglycolic acid, 3-mercaptopropionic acid, cysteine and homocysteine.

5. The coproduction process as claimed in claim 1, wherein said organic compound is selected from the group consisting of an amino acid bearing a thiol group, a peptide bearing a thiol group, mycothiol and dihydrolipoic acid .

6. The coproduction process as claimed in claim 1, wherein said organic compound is selected from the group consisting of: cysteine, homocysteine, glutathione, thioredoxin, mycothiol and dihydrolipoic acid, .

7. The coproduction process as claimed in claim 1, wherein said enzyme E is an oxidoreductase, .

8. The coproduction process as claimed in claim 1, wherein said enzyme D is a reductase or a dehydrogenase. .

9. The coproduction process as claimed in claim 1, wherein said sulfide is of general formula: R₁-S-R₂ in which,
R₁ and R₂ may be identical or different and are selected, independently of one another, from the group consisting of:
(C₁-C₂₀)alkyl, (C₂-C₂₀)alkenyl, (C₂-C₂₀)alkynyl, (C₃-C₁₀)cycloalkyl and (C₆-C₁₀)aryl or
R₁ and R₂ form, together with the sulfur atom to which they are attached, a heterocycloalkane or a heteroarene;
it being possible for said alkyl, alkenyl, alkynyl, cycloalkyl, aryl, heterocycloalkane and heteroarene groups optionally to be substituted by one or more substituents;
and it being possible for said alkyl, alkenyl, alkynyl, cycloalkyl and aryl groups to comprise one or more heteroatoms.

10. The coproduction process as claimed in claim 9, wherein the radicals R₁ and R₂ of said sulfide are identical.

11. The coproduction process as claimed in claim 1, wherein said sulfide is selected from the group consisting of dimethyl sulfide, diethyl sulfide, dipropyl sulfide, dibutyl sulfide, dioctyl sulfide, didodecyl sulfide and tetrahydrothiophene .

12. The coproduction process as claimed in claim 1, wherein the oxidizing agent is selected from the group consisting of air, oxygen-depleted air, oxygen-enriched air and pure oxygen.

13. The coproduction process as claimed in claim 1, wherein the cofactor is selected from nicotine cofactors and flavin cofactors.

14. The coproduction process as claimed in claim 1, wherein:
the sulfide is dimethyl sulfide;
the organic compound bearing at least one thiol group is glutathione;
the enzyme E is a Baeyer-Villiger Monooxygenase (BVMO) ;
the enzyme D is a glutathione reductase; and
the cofactor common to the two enzymes E and D is NADP.

15. A composition comprising:
1) a sulfide,
2) optionally an oxidizing agent,
3) an organic compound bearing at least one thiol group,
4) an enzyme E catalyzing the oxidation of said sulfide to sulfoxide or to sulfone,
5) an enzyme D catalyzing the formation of a disulfide bridge between two equivalents of said organic compound bearing at least one thiol group to form a dimer,
6) a cofactor common to the two enzymes E and D, and
7) a mercaptan.

16. A method for regenerating or recycling a cofactor used in the enzymatic oxidation of a sulfide to sulfoxide or to sulfone, the method comprising contacting the cofactor with a mercaptan.

17. The method as claimed in claim 16, where a disulfide bridge formed between two equivalents of an organic compound bearing at least one thiol group is reduced .

18. The method as claimed in claim 17, wherein the mercaptan is methyl mercaptan and the organic compound is glutathione.
